(19) United States
(12) Patent Application Publication
(10) Pub. No.: US 2008/0171949 A1
(43) Pub. Date: Jul. 17, 2008

(54) INTRAOPERATIVE ELECTROMAGNETIC APPARATUS AND RELATED TECHNOLOGY

(75) Inventors: Monica Silverstone Spiteri, Stoke-on-Trent (GB); Serguei Yurievich Semenov, Stoke-on-Trent (GB)

Correspondence Address: ADAMS INTELLECTUAL PROPERTY LAW, P.A., Suite 2350 Charlotte Plaza, 201 South College Street, CHARLOTTE, NC, 28244, US

(73) Assignee: The University Hospital of North Staffordshire NHS Trust, Stoke-on-Trent (GB)

(21) Appl. No.: 11/624,383

(22) Filed: Jan. 18, 2007

(30) Foreign Application Priority Data
Jan. 17, 2007 (GB) 0700873.3

(51) Int. Cl. A61B 5/05; A61N 1/00

(52) U.S. Cl. 600/547; 607/115

(57) ABSTRACT

An apparatus capable of applying a first electromagnetic signal, e.g. an optical signal, to a tissue portion is described. The apparatus is also capable of applying a second electromagnetic signal from a different portion of the electromagnetic spectrum, e.g. a microwave signal, to the tissue portion. Signals returned from the tissue portion and related to the first electromagnetic signal are analysed to give a first evaluation of the physical and biophysical characteristics of the tissue portion. Signals returned from the tissue portion and related to the second electromagnetic signal are also analysed, and a final evaluation of the physical and biophysical characteristics of the tissue portion is produced, informed by the first evaluation and the analysed second signal.

The present invention relates to methods and apparatus for of evaluating physical and biophysical characteristics of a tissue portion, to methods of determining disease state in a tissue portion, to methods of reconstructing a representation of a tissue portion, and to methods of treating a diseased part of a tissue portion, said apparatus and methods suitable for intraoperative diagnosis and treatment relating to tumour resections and general surgery on in vivo and ex vivo tissue portions.

### BACKGROUND OF THE INVENTION

Healthcare treatment spending continues to rise worldwide as providers invest in better diagnostic technologies and more efficacious therapeutic strategies to enhance disease prognosis and survival rates, whilst reducing adverse incidents.

In the field of healthcare there remains a need for healthcare professionals to obtain real time or near-real time confirmation of the patho-biological status of a particular target tissue, for example a surgeon may need such confirmation during the course of an operation.

When performing conventional excision biopsies and/or resection surgery from a potentially diseased tissue, it is often critical that the surgically removed sample is truly representative of the pathological state for which the procedure is intended. This is particularly the case for cancer patients. In the management of cancer patients, the prime clinical drivers are:

(1) to make surgery more accessible to patients diagnosed with cancer, or else suspected of having developed tumours;

(2) to provide accessible and practical sensing/imaging for tumour staging preferably at operation; and

(3) to reduce post-operative tumour recurrence and morbidity.

Surgical resection remains the basis for curative treatment in the majority of malignant tumours (cancers), provided that complete tumour resection is performed. In practice the precision with which complete tumour resection can be achieved may be influenced by anatomical and functional factors, but also by the ability or otherwise to evaluate microscopic tissue status at operation. As will be appreciated, the precision with which it is possible to distinguish normal tissue from diseased tissue during surgery is important in many other types of surgery. For example, in cardiac surgery it is desirable to distinguish normal myocardium tissue from ischemic/infracted/scar tissue.

Returning to the issue of tumour removal in cancer patients, it is currently accepted that lung cancer has one of the lowest survival rates of any cancer. Worldwide, lung cancer is the most common cancer with 1.3 million new cases diagnosed each year. In the United States, lung cancer is the leading cancer killer in both men and women, surpassing breast cancer to become the leading cause of cancer deaths in women. An estimated 163,510 deaths from lung cancer occurred in the United States during 2005. Between 1979 and 2002 lung cancer deaths increased by 60%. There were 38,000 new cases in 2000 in the UK, with 34,000 deaths that year. Lung cancer is currently responsible for 20% of all cancer deaths in the UK. In England and Wales ˜25% of patients survive one year, and ˜7% five years. Surgery can increase survival rates, raising the 5 year survival rate to more than 60% in cases where the disease is identified and treated at a very early stage.

There are currently no widely available effective intraoperative diagnostic modalities that can reliably inform and guide the surgeon in accurately evaluating tissue status at operation. Conventional surgical practice is to examine frozen sections removed during surgery, but this typically causes unnecessary operation time delays. Alternatively, the extent of tissue resection/excision can be determined at operation by macroscopic assessment and tactile perception. These practices commonly result in removal of more healthy tissue than is necessary, with possible subsequent postoperative structural deformity e.g. in breast, and unnecessary reduction in residual organ function e.g. in lung.

Another situation where tumour removal by surgery is commonplace is in treatment of breast cancer patients. Breast cancer is the second leading cause of cancer deaths in women today (after lung cancer) and is the most common cancer among women, excluding non-melanoma skin cancers. According to the World Health Organization, in excess of 1.2 million people are diagnosed with breast cancer each year worldwide, accounting for a tenth of all new cancers and 23% of all female cancer cases. Around 361,000 new cases of breast cancer occur each year in Europe. The American Cancer Society estimates that about 213,000 women in US are currently diagnosed with invasive breast cancer each year. The chance of developing invasive breast cancer during a woman's lifetime is approximately 13%. In the US it is estimated that another 62,000 women each year are diagnosed with in situ breast cancer, a very early form of the disease.

Breast conservation is an important aim in targeted breast cancer treatment, but is dependent on early detection, and on reliable diagnostic feedback at operation. Frozen section analysis which is routinely used in breast tumour surgery to determine diagnosis and extent of resection can be difficult to perform and may provide inconclusive results. As with lung cancer and indeed other organ tumours, surgical care for breast tumours would be greatly enhanced by availability of precise immediate tissue status feedback intraoperatively.

Development and use of diagnostic modalities that can be used for tissue screening within the clinical setting of a surgical suite would allow patients to obtain more effective, accurate surgical care, in a shorter time interval and with likely reduced postoperative morbidity and tumour recurrence.

Tremendous advances in medical interventions have been made to assist patient screening and pre-operative staging of cancer, specifically to enhance early diagnosis, disease staging and optimization of surgical provision e.g. low-dose spiral computed tomography; positron emission tomography and magnetic resonance imaging. These technologies are significantly better than previous conventional diagnostic approaches such as chest radiograph and standard computed tomography.

However these modalities are inherently costly to install and maintain, and are therefore limited in their availability. This leads to discrepancies in geographic availability, and may be responsible for significant time lags in patient access to diagnostic screening and the associated feedback to clinician. Some of these modalities use ionizing radiation which presents potential hazard. Also, because of their physical bulk they may not be practical for surgical suite or clinical room installation and use. In respect of tumour imaging and screening, there remain concerns of associated over diagnosis and false positive test results leading to unnecessary treatments and heightened patient anxiety.

Other technological advances in bio-imaging modalities include: optical and ultrasound mammography; optical imaging including optical coherence tomography; electrical impedance, microwave and terahertz imaging and tomography; endobronchial ultrasound; and fluorescent imaging. However, these modalities are generally in early stage research or at the pre-clinical trial stage.

There is therefore a need for development and provision of a consumer-friendly, versatile, diagnostic modality suited to the surgical theatre, incorporating the practical requirements of being mobile and low maintenance such that it can conveniently be made available for use at the point of care. This modality should provide the surgeon with real time or near-real time identification of any morphological/functional changes of the target tissue, and provide diagnostic feedback on the patho-biological status of the target organ and/or an excised sample thereof. Such a modality would avert need to perform frozen tissue processing or staining, and reduce post-operative invasive interventions such as further operations. The advent of such a modality would be a major advance in intraoperative care provision.

It is an aim of preferred embodiments of the present invention to address one problem associated with the prior art, whether identified herein, or otherwise.

According to the present invention there is provided an apparatus and method as set forth in the appended claims. Preferred features of the invention will be apparent from the dependent claims, and the description which follows.

### BRIEF DESCRIPTION OF THE DRAWINGS

For a better understanding of the invention and to show how embodiments of the same may be put into effect, the various aspects of the invention will now be described by way of example only with reference to the accompanying diagrams, in which:

FIG. 1 illustrates a schematic diagram of an electromagnetic imaging apparatus according to a first exemplary embodiment of the present invention including a computer and a visual display unit;

FIGS. 2A-2E show front cross sectional views of probe heads for use in the apparatus of FIG. 1;

FIG. 3 shows a side sectional view of a tip of a probe head for use with the embodiment of FIG. 1, with the associated electromagnetic field distribution; and

FIG. 4 shows an example flow diagram illustrating how embodiments of the invention may function in a clinical setting.

### DESCRIPTION OF THE PREFERRED EMBODIMENTS

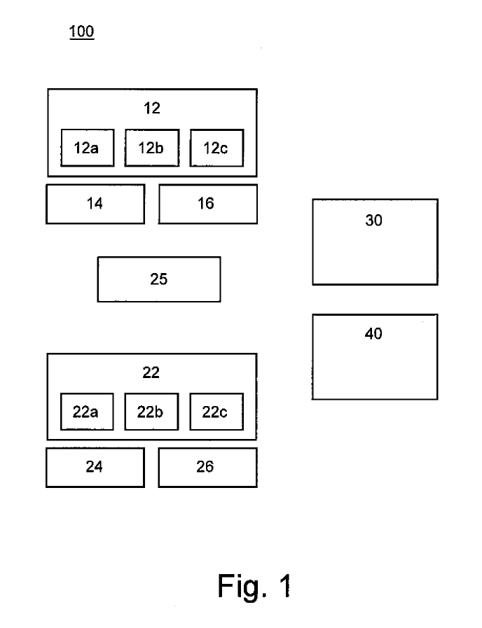

FIG. 1 shows a schematic block diagram of an apparatus 100 for evaluating physical and biophysical characteristics of a tissue portion according to a first exemplary embodiment of the present invention. The apparatus 100 comprises first and second electromagnetic signal generating units 12,22, first and a second electromagnetic signal detecting units 14,24, and first and second electromagnetic signal analysing units 16,26.

The first electromagnetic signal generating unit 12 comprises a source of electromagnetic radiation of a first portion of the electromagnetic spectrum, and the first electromagnetic signal detecting unit 14 is operable to detect electromagnetic radiation of the first portion of the electromagnetic spectrum, such as radiation returned from a tissue portion after such radiation has been applied to the tissue portion from the first electromagnetic signal generating unit 12. The first electromagnetic signal analysing unit 16 is operable to give a first evaluation of physical and biophysical characteristics of the tissue portion based on knowledge of the electromagnetic radiation output by the first electromagnetic signal generating unit 12 that is applied to the tissue portion, and based on the electromagnetic radiation detected by the first electromagnetic detecting unit 14.

The second electromagnetic signal generating unit 22 comprises a source of electromagnetic radiation of a second portion of the electromagnetic spectrum, and the second electromagnetic signal detecting unit 24 is operable to detect electromagnetic radiation of the second portion of the electromagnetic spectrum returned from a tissue portion. The second electromagnetic signal analysing unit 26 is operable to analyse the electromagnetic radiation detected by the second electromagnetic detecting unit 24.

The apparatus 100 further comprises a final evaluation unit 25 operable to produce a final evaluation of the physical and biophysical characteristics informed by the first and second electromagnetic signal analysing units 16, 26 and analyses. Also shown in FIG. 1 is a computer 30, and a visual display unit 40 as a user interface portion of the apparatus 100. The final evaluation produced by the final evaluation unit 25 can be provided to the visual display unit 40 for display, or to the computer 30 for further analysis.

By combining information relating to the first electromagnetic signal generating and detecting units 12,14 with information from the second electromagnetic signal generating and detecting units 22,24 the final evaluating unit 25 can provide an improved output over that possible with the first and second analysing units 16,26 alone. Further on, by intermediate and parallel combination of information provided by the first electromagnetic signal analysing unit 16 and the second electromagnetic signal analyzing unit 26 may provide an improved performance of each of analysing units. For example, if an iteration analysing procedure is used, then results of an ith iteration from the first electromagnetic signal analysing unit 16 might be used as a correction/adjustment information at jth iteration in the second electromagnetic signal analysing unit 26 and vise versa.

As the inventors have appreciated, electromagnetic radiation from a first portion of the electromagnetic spectrum will interact with a given type of tissue portion in a different way to electromagnetic radiation from another portion of the electromagnetic spectrum. For example, in biological tissues microwaves will generally penetrate a greater distance than radiation of optical frequencies. Light waves delivered to a portion of tissue to be imaged reflect off the internal structural layers within the scanned section, allowing micron-scale resolution capable of distinguishing normal anatomy and in situ morphological aberrations. The morphology of individual anatomical components varies in relative thickness, cellular composition and density as well as relative amount of cellular/extracellular matrix, as revealed by standard histological analysis. This results in inherent different optical properties, such as optical scattering, reflection and transmission.

The shorter wavelength of radiation in the optical frequency range means that greater resolution can be achieved in an image constructed using optical techniques as compared to one constructed using microwave techniques. Despite the inherently lower resolution of microwave techniques as compared with optical, it may be very useful to evaluate the physical and biophysical characteristics of a tissue portion over a deeper range than can be achieved with optical techniques.

Tissue characteristics of particular interest in relation to the low frequency to microwave portions of the electromagnetic spectrum are the dielectric properties of tissues; the real and imaginary parts of the dielectric permittivity are known to vary dependent on physiological properties of tissues in these portions of the electromagnetic spectrum. Tissue dielectric properties reflect a variety of tissue characteristics and composition properties, such as extracellular, membrane and intracellular conductivity and permittivity; cellular, protein and water (bound and free) volume fraction; and ion conductivity.

Tissue characteristics of particular interest in relation to the optical portions of the electromagnetic spectrum are the optical scattering and absorption coefficients, attenuation, trasmittance and reflection. However, other optical techniques or properties may also be used in the evaluation of the physical characteristics of the tissue portion.

Based on this, the first portion of the electromagnetic spectrum used in the apparatus of FIG. 1 may comprise the optical portion or the optical to infrared portion, and the second portion of the electromagnetic spectrum may comprise the low frequency to microwave portion. Within these portions, the signals applied may be of a single frequency, or a range of frequencies. The signals applied may also vary in frequency over time as they are applied, e.g. as swept frequency signals, or a number of discrete sampling signals of different frequencies may be used simultaneously.

A suitable processing technique for analysing the detected signals in reconstructing the characteristics of interest may be based on using an inverse problem algorithm to iteratively converge on a solution from an initial guess. However, such processing is relatively computationally intensive, and in some circumstances may diverge away from the actual solution. However, in example embodiments of the present invention information from the first electromagnetic signal analysing unit 16 can inform the inverse problem algorithm for producing the final evaluation by feeding into the final evaluation unit 25, for example at an early stage of producing the final evaluation. Knowledge of the optical properties of the tissue portion of interest established by the first signal analysing unit 16 can be used to help in selecting the initial guess for the inverse problem algorithm and vice versa for dielectric properties and second electromagnetic unit 26. A small improvement in the accuracy of the initial guess and or during an iterative procedure can reduce the chance of diverging away from an actual solution, and can vastly reduce the number of iterations required to achieve a desired level of convergence. In the example embodiment of FIG. 1 the optical properties in the tissue portion may give an indication of the type of material in the tissue portion, which can be used to guide an initial guess and/or at an ith iteration for the dielectric properties of the tissue portion determined from the second electromagnetic signals, as well as in the final evaluation. Respectively, in the example embodiment of FIG. 1 the dielectric properties in the tissue portion may give an indication of the type of material in the tissue portion, which can be used to guide an initial guess and/or a jth iteration for the optical properties of the tissue portion determined from the first electromagnetic signals, as well as in the final evaluation.

A number of procedures may be used in the analysing units, for example in the optical and/or infrared portions of the electromagnetic spectrum a light propagation approximation of the Burger type may be appropriate in analysing the optical signal returned from the tissue portion. Alternatively or in addition procedures based on radiation transfer theory and/or Monte-Carlo optimisation may be appropriate. In the low frequency and/or microwave portions of the electromagnetic spectrum a Born and Rytov approximation may be appropriate, or alternatively a Helmholtz wave equation method may be used with Newton and/or Gradient inversion and Tikhonov regularisation.

The apparatus 100 is intended for use in the evaluation of tissue portions, particularly in vivo or ex vivo tissue portions. Such evaluation can allow the apparatus to characterize and distinguish normal functional morphology from abnormal dysfunctional and pathobiological states, thereby providing a tool which can be used to guide the diagnosis of any departure from health and normality. Of particular utility in this field is the ability to distinguish malignant tumours (cancer) from benign lesions. By analysing dielectric and optical properties of a target tissue portion and patterns of electromagnetic and optical fields distribution within the tissue diagnostic information such as whether the tissue portion comprises (i) healthy normal morphological tissue, or (ii) any in situ functional morphological changes consequent on presence of inflammation, infection, ischemia, tissue blood content, neovascularisation, infarction, fibrosis, benign cyst formation and/or malignant transformation may be obtained.

To follow the evaluation through to diagnosing a clinical disease state, steps involving relating the dielectric and optical properties of particular tissues to their health, viability and pathobiological states as defined by conventional methods of molecular biology, immunocytochemistry and immunohistology of same tissue may be performed. These steps will enable databases of dielectric and optical parameters of in vivo or ex vivo tissues to be compiled. The databases may include characterized differences across and between types of normal tissue in terms of function and morphology as a so-called “spectrum of predicted normal status”. Furthermore, the databases may include distinguishing features from pathobiological states as listed above.

In preferred embodiments, the database of optical and/or dielectric properties of target tissue types may be stored on the computer 30. The optical properties of interest may include transmission, reflectance, scattering including g-factor and absorption characteristics of the tissues or microstructures within the tissue. In particularly preferred embodiments, means to compare optical and/or dielectric properties of a target tissue type, as examples of the physical characteristics evaluated by the apparatus 100 with the database of tissue properties in the database are present in the computer 30. Preferably the computer 30 also includes means to indicate to a user when a potential match between the evaluated optical and/or dielectric properties of a tissue or microstructure within a tissue has been matched with optical and/or dielectric properties of a specific tissue type in the database. Such means may include use of the display unit 40, or an audible signalling unit (not shown).

The apparatus may evaluate a tissue portion at a time-sampling mode of e.g. 20-1000 samples per second. In order to reduce the overall computational load involved in dealing with the information generated, it may be possible to prioritise evaluation depending on a degree of confidence associated with initial evaluation results, and/or predetermined characteristics associated with initial evaluation results. For example, the apparatus may be configured to provide an evaluation of tissue portions showing signs of a particular predetermined disease state. If initial evaluation results show that a particular tissue portion is likely to correspond to the predetermined disease state the frequency, intensity and/or resolution of the apparatus' operation may be increased to maximum or near maximum levels, and the most accurate processing techniques available may be used.

In example embodiments it may be advantageous for there to be a degree of physical overlap of the area or volume of the tissue portion into which the first and second electromagnetic signals are applied, as this is likely to give a higher chance that the first evaluation is useful in producing the final evaluation at first and/or subsequent iteration.

In the example embodiment of FIG. 1 it may be advantageous for the first signal generating unit 12 to comprise a first signal generator 12a coupled by a first signal transmission portion 12b to a first signal outlet 12c, and for the second signal generating unit 22 to comprise a second signal generator 22a coupled by a second signal transmission portion 22b to a second signal outlet 22c. The signal outlets 12c,22c may be arranged to deliver electromagnetic energy from a probe head, closely positioned or integrated with one another in or around the probe head. This arrangement can give a high degree of overlap between parts of the tissue portion the first and second signals are applied to. This arrangement is also suitable for incorporation in to an endoscope or similar device.

The first signal transmission portion 12b may suitably comprise an optical fibre, whereas the second signal transmission portion 22b may suitably comprise a co-axial cable.

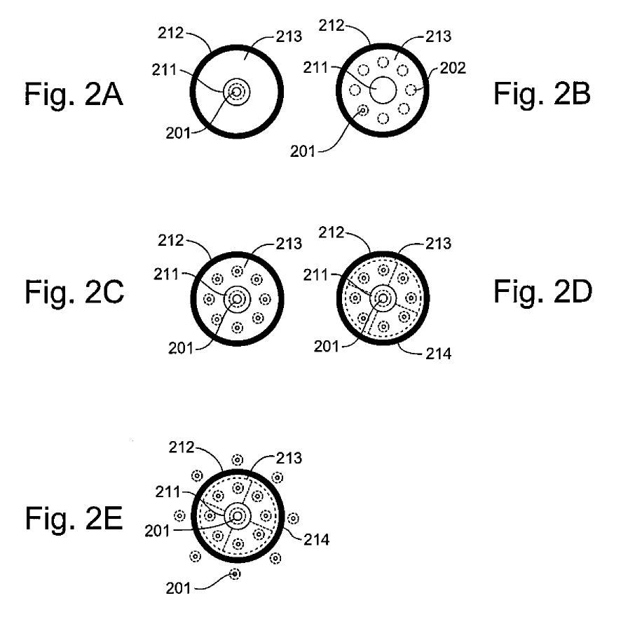

FIGS. 2A-2E each show a cross sectional view of an example probe head. FIG. 2A shows a centrally positioned optical fibre 201 used to transmit and receive optical signals. The optical fibre 201 is surrounded by an electrical conductor in the form of a first metal portion 211, and by an electrical conductor in the form of a second metal portion 212 spaced apart from and surrounding the first metal portion 211. The first and second metal portions 211,212 form a co-axial cable, and are conveniently maintained in a spaced apart relationship by a suitable dielectric 213 positioned there between.

FIG. 2B shows a cross sectional view of a second example probe head which can be used with embodiments of the present invention. FIG. 2B shows a centrally positioned electrical conductor in the form of a first metal portion 211, surrounded by an electrical conductor in the form of a second metal portion 212 spaced apart from and surrounding the first metal portion 211. The first and second metal portions 211,212 form a co-axial cable, and are conveniently maintained in a spaced apart relationship by a suitable dielectric 213 positioned there between.

In this probe head an optical fibre 201 is positioned in the dielectric 213 and is used to transmit and receive optical signals. The optical fibres 202 distributed elsewhere in the dielectric in a rotationally symmetric pattern are used to receive optical signals. Other arrangements, such as the use of asymmetrically distributed fibres may be particularly useful if the optical polarisation properties are to be evaluated.

The probe heads arrangements shown in FIGS. 2A and 2B allow the apparatus to conveniently transmit and receive signals which when analysed may provide a measurement of optical and dieletric (scalar or complex) reflection coefficients of the tissue portion to which the probe head is applied.

FIG. 2C shows a cross sectional view of a third example probe head including features of the probe heads of FIGS. 2A and 2B, but in which all of the optical fibres 201 shown may be used to transmit and receive optical signals. FIG. 2D shows a cross sectional view of a fourth probe head in which additional electrodes 214 are provided in the dielectric 213. FIG. 2E shows a cross sectional view of a fifth probe head in including all the features of the probe head of FIG. 2D, but with an additional ring of optical fibres 201 around the outer periphery of the second metal portion 212.

The probe head arrangements shown in FIGS. 2C-2E allow the apparatus to conveniently transmit and receive signals which can be used in a tomographic evaluation. All of the embodiments may be used to evaluate in one dimension along the axis of the probe head; in two dimensions, both axially and radially to the probe head; or in three dimensions such as by using axial distance, radial distance and an angle in a cylindrical coordinate system.

Although the probe heads shown in FIGS. 2A-2E are all of the same general type, it is to be understood that other geometric arrangements of sources and detectors are possible, e.g. square or rectangular. It is also to be understood that both the microwave and optical waves may be delivered by other means.

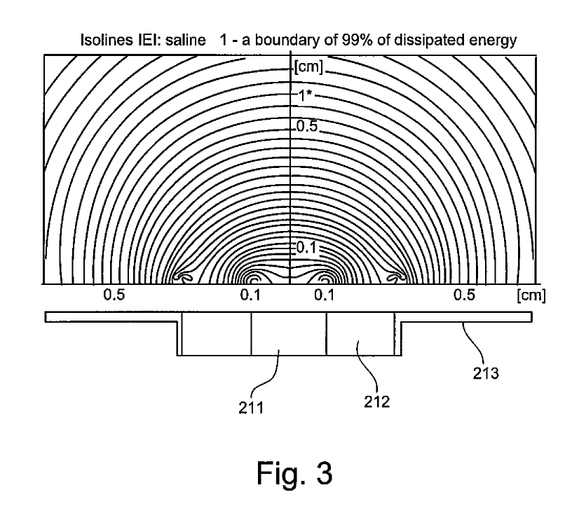

FIG. 3 shows the tip of the probe head of similar configuration to that of FIG. 2A in a side cross-sectional view. Also shown in FIG. 3 is a schematic representation of the typical electromagnetic field distribution near the tip of the probe head in the low frequency to microwave portions of the electromagnetic spectrum.

In a further embodiment of the present invention a method of reconstructing a representation of a tissue portion is provided. In the method a final evaluation of physical characteristics of the tissue portion is performed as above. The evaluated physical and biophysical characteristics are compared to previously established model of physical properties of tissue types, and a reconstructed representation of the tissue portion is established based on the comparison of the final evaluation and the model.

In more detail, the physical characteristics of tissues in the low frequency to microwave portions of the electromagnetic spectrum, e.g. tissue dielectric properties depend on a variety of tissue composition properties, including intracellular, extracellular, vascular and membrane resistance and capacitance. Other relevant factors are cellular volume fraction, geometric shapes, compositions and interconnections, water and proteins volume fractions. A model of these tissue dielectric properties as a complex superposition of the free water, bound water, protein and cell relaxation processes with corresponding volume fractions and the frequency independent ion conductivity has been developed, and is expressed in Equation 1 below.

$${ɛ}_{tissue} = {\sum }_{m} {ɛ}_{cell} ( f ) + {ɛ}_{iR} + ( {ɛ}_{0} - {ɛ}_{iR} ) {\sum }_{i = 1}^{N} \frac{{K}_{i}}{1 + j ( f / {f}_{i} )} - j \frac{{σ}_{ion}}{2 π f {ɛ}_{v}} \qquad Equation 1$$

where:
N=3
j²=−1
K₁=K_{W};f₁=f_{w}—free water volume fraction and relaxation frequency
K₂=K_{bw}; f₂=f_{bw}—bound water volume fraction and relaxation frequency
K₃=Kₚᵣ; f₃=fₚᵣ—major protein volume fraction and relaxation frequency
ε₀; ε_{iR}=permittivities at low (near zero) and high (infrared) frequencies
σᵢₒₙ—ion conduction component
εᵥ=dielectric constant of vacuum
ε_{cell}=cellular relaxation component for mth type of cells

ε_{cell} can be expressed in a simplified form as shown in Equation 2, though a more complex representation is needed when electrical interactions between cells are taken into account.

$${ɛ}_{cell} = {ɛ}_{2} + p {ɛ}_{2} d \frac{( {ɛ}_{1} - {ɛ}_{2} ) [ {ɛ}_{0} + ( d - 1 ) {ɛ}_{1} ] + ( {ɛ}_{0} - {ɛ}_{1} ) [ {ɛ}_{2} + ( d - 1 ) {ɛ}_{1} ] \frac{{r}_{0}^{d}}{{r}_{1}^{d}}}{\begin{matrix}[ {ɛ}_{0} + ( d - 1 ) {ɛ}_{1} ] [ ( {ɛ}_{1} + ( d - 1 ) {ɛ}_{2} ) + p ( {ɛ}_{2} - {ɛ}_{1} ) ] + \\ \frac{{r}_{0}^{d}}{{r}_{1}^{d}} ( {ɛ}_{0} - {ɛ}_{1} ) [ ( d - 1 ) ( {ɛ}_{1} - {ɛ}_{2} ) - p ( ( d - 1 ) {ɛ}_{1} + {ɛ}_{2} ) ]\end{matrix}} \qquad Equation 2$$

where:
p=volume fraction;

indexes 0,1 and 2 corresponds to intracellular, membrane and extracellular dielectric properties
r=cellular radius
d=2 for 2D and 3 for 3D approaches respectively

A functional minimization procedure can be used in searching for parameters of the model. Denoting all unknown parameters (for example: volume fractions, relaxation frequencies, dielectric properties 0,1 and 2 above etc.) of the model as a column xi (see Equation 3 below). A function of the xi parameters can be created as a normalized deviation between the evaluated physical characteristic “εexp” and a theoretically predicted value “εthr” of the model. This is represented by Equation 3.

$$F ( {x}_{1} , … , {x}_{N} ) = \frac{1}{{N}_{p t}} \sqrt{{\sum }_{t = 1}^{{N}_{p t}} {( {ɛ}_{exp}^{ret} - {ɛ}_{thr}^{ret} )}^{2}} + \frac{1}{{N}_{p t}} \sqrt{{\sum }_{t = 1}^{{N}_{p t}} {( {ɛ}_{exp}^{img} - {ɛ}_{thr}^{img} )}^{2}} \qquad Equation 3$$

where:
N=the number of parameters to be found
Nₚₜ=the number of the experimental spectral points.

The above function can be minimized using a number of methods either alone or in combination. Such methods include coordinate slopes on the N parameters; golden section search and sorting method on each parameter; and gradient methods.

In this way the apparatus is operable to evaluate the physical and biophysical characteristics of a tissue portion, and in addition to reconstruct a representation of the tissue portion. In the same way as the apparatus may compare the evaluated physical characteristics against a database of tissue properties, it may also be possible to automatically recognise tissue functional morphological patterns in the reconstructed representation of the tissue portion, and to use this to signal appropriate status to the operator, thereby averting need for detailed operator interpretation. In this way the potential problems of operator interpretation bias, lengthy training times and possible consumer apprehension to new medical devices/change in care provision are avoided.

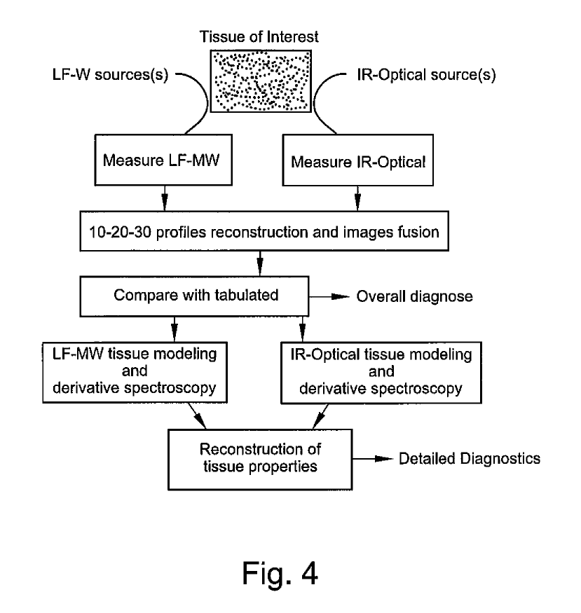

FIG. 4 shows an example flow diagram illustrating how embodiments of the invention may function in a clinical setting.

In example embodiments the apparatus may operate in response to, or in synchronisation with some characteristics of the tissue portion of interest. For example, if the tissue portion of interest is part of a living organism, the apparatus may operate in synchronisation with a heart beat or respiratory cycle of the organism. This technique may lead to improved evaluation of the physical characteristics of the tissue portion. Monitoring for tumorogensis and early stage cancers using indicators associated with neovascularisation and vascularo-genesis can be made simpler and more effective by synchronisation/circulation gating techniques. Furthermore, monitoring circulation in a synchronised/circulation gated system may give additional information in respect of which tissue areas are diseased; it is recognised that cancer cells typically have greater blood supply than corresponding healthy cells.

Using the apparatus and methods described above the reader will appreciate that it becomes possible to provide near-morphological/functional information on sub-surface target tissues and/or tissues specimen in real-time and/or near-real time. This allows a user to characterise the pathobiological status of tissue, and so generate a clinical diagnosis. In cases of cancer, it may therefore become possible to determine the presence and extent of primary tumour/tumours, grade and invasiveness, in situ satellite lesions, site lymph node involvement, intracavitary disease involvement such as within thorax, abdomen, pelvis, etc.

Once the physical/biophysical properties of the tissue have been evaluated, precision surgery, especially in cases of resection, providing precise definition of negative resection margins at both the proximal and distal ends of the surgical organ/tissue resection is facilitated. Resection is a procedure that involves removal of the tumour and appropriate amount of the organ, which may be whole or partial, lobar or sublobar or segmental or sleeve, depending on the particular organ type, dependent on clinical need and status of patient and tumour nature and/or site. Resection may also be required in pathological situations other than tumour diagnosis for example in cases of ischemia and infarction of tissue and/or organs e.g. intestine, bowel and also in cases of abnormal vascular supply to the limbs, ischemic limbs through vascular occlusive disease and during elective and emergency limb amputations. Surgical intervention might also be required in trauma/extremities injuries, for example for treatment of compartment syndrome.

With particular reference to cancer care, the provision of intra-operative feedback on the physical characteristics of tissue will dramatically reduce the time delay between surgical operation for removal of the tumour and diagnostic feedback on the status of tumour excised, and on the presence or otherwise of negative free margins. This has a direct bearing on informed need for additional therapeutic procedures such as systemic chemotherapy and/or radiotherapy. The apparatus may in example embodiments by used as a guide to adjunct therapy as necessary at time of operation. This is particularly important, as the device described herein may be used to provide in-situ ablation. The device may be used, either under manual control or under automatic control to apply intense electromagnetic radiation to the tissue in question to destroy diseased cells. The electromagnetic radiation used in this way may be one or both of microwave and optical, and may be provided from/by the apparatus described above in relation to the evaluation of tissue portions. Other electromagnetic radiation may also be provided in addition or as an alternative to the electromagnetic radiation used in evaluating tissue portions.

The device may also be useful in the provision of brachytherapy and/or targeted chemotherapy, or simple resection by cutting. The provision of adjunct therapy at time of operation which is facilitated by the apparatus is likely to improve and enhance outcomes for cancer patients and reduce post-operative morbidity, particularly in high risk patients.

During an operation a chain of: diagnosis→identification of target area of tissue→ablation using microwave and/or optical radiation of thermogenic intensities from the same device→feedback on the efficiency of ablation using diagnostic capabilities of the same device can be performed with further abaltion or adjunct therapies then provided if needed.

The apparatus makes possible a “one-stop shop” intra-operative approach, which would reduce patient anxiety and unnecessary time delay to vital treatment, reduce unnecessary waiting times and also reduce potential service costs such as personnel, extra clinical sessions as is often required in conventional approaches to cancer post-operatively.

The apparatus may extend the usefulness of surgical procedure for patients with other forms of cancer to be offered the surgical option, even those patients with cancer who may be poor candidates for the ideal surgical intervention, due to poor health or other medical or technical issues. The apparatus enables an informed surgical approach to management of their cancer to be adopted, in which resectability versus operability can be objectively balanced, allowing that even in situations where subtotal resections have to be performed in order to preserve organ function, additional adjunct therapy to the target site such ablation, brachytherapy and/or in situ chemotherapy can be provided. In this way, post-operative disease recurrence may be reduced and optimal organ/tissue function retained. These factors may make a significant difference in outcome and survival by facilitating precision, organ conserving surgery.

The apparatus described herein, and the associated methods can be used to provide accurate, real time or near-real time determination of a tissue type and status in an intra-operative or similar setting. This can facilitate diagnosis of the functional and morphological status of tissue, thereby empowering precision tissue resections and guiding appropriate intra-operative adjunct therapy. The apparatus is widely suitable in situations where it is desired to evaluation tissue in organs such as the lung, breast, colorectal, gastrointestinal/stomach/oesophagus, brain, prostate, eye, skin and any other tissue (including lymph nodes), in particular those likely to give rise to malignant transformation and where resection may be required. The apparatus may be applicable to both adult and paediatric diseases and procedures. Furthermore, the apparatus may be useful during conventional excision biopsies and/or resection surgery from a potentially diseased tissue, to evaluate the characteristics of a surgically removed tissue sample/specimen as truly representative of the pathological state for which the procedure is intended. In particular the apparatus may be able to evaluate local changes invisible to the naked eye.

As further mentioned above, the apparatus can be used outside the operating suite to facilitate more general disease screening. The apparatus can be used in bronchoscopy, thoracoscopy, mediastinoscopy, laparoscopy, colonoscopy, sigmoidoscopy, colposcopy, cystoscopy, arthroscopy. As well as such in vivo uses, the apparatus has utility with ex vivo samples, as such expediting histological processing and analysis and maximise throughput within, pathology laboratories etc., allowing rapid diagnostic and/or screening differentiation of specimens/samples, potentially reducing conventional associated costs in labour and reagents.

The embodiments described above are generally applicable to animal tissues, and may be useful in both medical and veterinary spheres.

Although a few preferred embodiments have been shown and described, it will be appreciated by those skilled in the art that various changes and modifications might be made without departing from the scope of the invention, as defined in the appended claims.

The reader's attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings), may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiments). The invention extends to any novel one, or any novel combination, of the features disclosed in this specification (including any accompanying claims, abstract and drawings), or to any novel one, or any novel combination, of the steps of any method or process so disclosed.

1. A method of evaluating physical and biophysical characteristics of a tissue portion, the method comprising the steps of:
applying first and second electromagnetic signals to the tissue portion, the first and second electromagnetic signals from different portions of the electromagnetic spectrum;
analysing a first signal returned from the tissue portion and related to the first electromagnetic signal to give a first evaluation of the physical characteristics;
analysing a second signal returned from the tissue portion and related to the second electromagnetic signal; and
producing a final evaluation of the physical characteristics informed by the first evaluation and the analysed second signal.

2. The method of claim 1, wherein the step of analysing the first and second signals returned from the tissue portion is informed by knowledge of the electromagnetic signals applied to the tissue portion.

3. The method of claim 2, wherein the first electromagnetic signal is from the optical portion and/or infra-red portion of the electromagnetic spectrum.

4. The method of claim 3, wherein the first electromagnetic signal has a wavelength of 0.4 μm to 4 μm.

5. The method of claim 2, wherein the second electromagnetic signal is from the low frequency and/or microwave portion of the electromagnetic spectrum.

6. The method of claim 5, wherein the second signal has a frequency of 0.1 MHz to 10 GHz.

7. The method of claim 1, wherein the first and second electromagnetic signals are applied to at least partially overlapping physical areas or partially overlapping physical volumes of the tissue portion.

8. The method of claim 1, further comprising providing a user with a visible and/or audible output representative of one or more characteristics of the final evaluation.

9. The method of claim 1, wherein the first and/or second electromagnetic signals have one or both of: time-varying frequency; and a plurality of signals at different frequencies transmitted and analysed simultaneously, within the first and second portions of the electromagnetic spectrum.

10. Apparatus for evaluating physical and biophysical characteristics of a tissue portion, the apparatus comprising:
first and second electromagnetic signal generating units, first and a second electromagnetic signal detecting units, and first and second electromagnetic signal analysing units, wherein:
said first and second electromagnetic signal generating units are operable to apply first and second electromagnetic signals to the tissue portion, the first and second electromagnetic signals from different portions of the electromagnetic spectrum;
said first electromagnetic signal detecting unit is operable to detect and analyse a first signal returned from the tissue portion and related to the first electromagnetic signal to give a first evaluation of the physical characteristics;
said second electromagnetic signal detecting unit is operable to detect and analyse a second signal returned from the tissue portion and related to the second electromagnetic signal; and
the apparatus further comprising a final evaluation unit operable to produce a final evaluation of the physical characteristics informed by the first evaluation and the analysed second signal.

11. The apparatus of claim 10, wherein the analysis of the first and second signals returned from the tissue portion performed by the first and second signal analysing units is informed by knowledge of the of the electromagnetic signals applied to the tissue portion by the first and second signal generating units.

12. The apparatus of claim 11, wherein the first signal generating unit is operable to apply a first electromagnetic signal from the optical portion and/or infra-red portion of the electromagnetic spectrum.

13. The apparatus of claim 12, wherein the first signal generating unit is operable to apply a first electromagnetic signal from the portion of the electromagnetic spectrum having wavelength of 0.4 μm to 4 μm.

14. The apparatus of claim 10, wherein the second signal generating unit is operable to apply a second electromagnetic signal from the low frequency and/or microwave portion of the electromagnetic spectrum.

15. The apparatus of claim 14, wherein the second signal generating unit is operable to apply a second electromagnetic signal from the portion of the electromagnetic spectrum having frequency of 0.1 MHz to 10 GHz

16. The apparatus of claim 10, wherein the first and second signal generating units are operable to apply first and second electromagnetic signals to an at least partially overlapping physical area or partially overlapping physical volume of the tissue portion.

17. The apparatus of claim 10, wherein the first signal generating unit comprises a first signal generator coupled by a first signal transmission portion to a first signal outlet, and/or the second signal generating unit comprises a second signal generator coupled by a second signal transmission portion to a second signal outlet.

18. The apparatus of claim 17, wherein the first and/or signal outlets are integrated in a probe head.

19. The apparatus of claim 18, wherein the probe head is integrated with an endoscope.

20. The apparatus of claim 17, wherein the first signal transmission portion comprises an optical fibre.

21. The apparatus of claim 17, wherein the first signal generating unit comprises a plurality of first signal generators coupled by a plurality of first signal transmission portions to a plurality of first signal outlets.

22. The apparatus of claims 17, wherein one or more of the first transmission portions transmits a first signal returned from the tissue portion and related to the first electromagnetic signal to the first signal detecting unit.

23. The apparatus of claim 17, wherein the second transmission portion comprises a co-axial cable.

24. The apparatus of claim 17, further comprising one or more electrodes arranged to transmit a second signal returned from the tissue portion and related to the second electromagnetic signal to the second signal detecting unit.

25. The apparatus of claim 24, wherein the electrode or electrodes are arranged between the conductors of the co-axial cable.

26. The apparatus of claims 17, wherein, the arrangement of one or more of: the co-axial cable; the signal outlets and the electrodes in the probe head in cross section exhibit one or more of: circular; rotational; and reflective symmetry.

27. The apparatus of claim 10, further comprising a user interface unit operable to provide a visible and/or audible output representative of one or more characteristics of the final evaluation produced by the final evaluation unit.

28. A method of determining disease state in a tissue portion, the method comprising the steps of:
producing a final evaluation of physical characteristics of the tissue portion using the method of claim 1;
comparing the final evaluation to corresponding characteristics of samples in a library of reference samples of known disease state to find a closest match; and
determining a disease state of the tissue portion as that corresponding to the disease state of the reference sample which is the closest match.

29. A method of reconstructing a representation of a tissue portion, the method comprising the steps of:
producing a final evaluation of physical characteristics of the tissue portion using the method of claim 1;
comparing the final evaluation to a previously established model of physical properties of tissue types; and
reconstructing a representation of the tissue portion based on the comparison of the final evaluation and the model.

30. A method of treating a diseased part of a tissue portion, the method comprising:
applying electromagnetic ablation to the diseased parts of the tissue portion.

31. The method of treating a diseased part of a tissue according to claim 30, the method comprising the further step of: either (a) determining disease state or states in parts of the tissue portion using the method of claim 28; or (b) reconstructing a representation of the tissue portion and identifying in the reconstructed representation disease state or states in parts of the tissue portion.
